(19) United States
(12) Patent Application Publication
(10) Pub. No.: US 2026/0096886 A1
(43) Pub. Date: Apr. 9, 2026

(54) ONE WAY VALVE FOR BRONCHUS

(71) Applicant: BEIJING CARDIOTECH MEDICAL TECHNOLOGY CO., LTD, Beijing (CN)

(72) Inventors: Gang HOU, Beijing (CN); Ting YANG, Beijing (CN); Chen WANG, Beijing (CN)

(21) Appl. No.: 19/115,289

(22) PCT Filed: Sep. 19, 2023
(86) PCT No.: PCT/CN2023/119677

§ 371 Date: Mar. 26, 2025

(30) Foreign Application Priority Data
Sep. 26, 2022 (CN) 202211176959.1

(51) Int. Cl. A61F 2/24; A61F 2/04

(52) U.S. Cl. CPC A61F 2/2476; A61F 2002/043

(57) ABSTRACT

A one-way valve for a bronchus, comprising: a support portion, the outer wall of the support portion being used for abutting against the inner wall of a bronchus; and an air-permeable portion provided in the support portion and connected to the inner wall of the support portion. The support portion and the air-permeable portion are of an integrated structure. The technical solution in the prior art of a combination of a nickel-titanium alloy framework and a coating material can be replaced with the present invention, such that the production cost of the one-way valve for a bronchus can be reduced; moreover, the yield of one-way valves for a bronchus can be increased, and the popularization and use of the one-way valves for a bronchus can be facilitated.

The present application is a national stage entry of International Application No. PCT/CN2023/119677 filed Sep. 19, 2023, which claims the priority to a Chinese patent application 202211176959.1 filed with the China National Intellectual Property Administration (CNIPA) on Sep. 26, 2022 and entitled “ONE-WAY VALVE FOR BRONCHUS”, the disclosures of which are incorporated herein by reference in their entireties.

### TECHNICAL FIELD

The embodiments of the present application relate to the field of medical technology, and in particular to a one-way valve for bronchus.

### BACKGROUND

Chronic obstructive pulmonary disease is a common chronic respiratory disease with high morbidity and mortality. Patients with COPD with severe emphysema phenotype are insensitive to drug therapy, and a lung volume reduction surgery and a lung transplantation have become important treatment measures for such patients. Currently, a bronchoscopic lung volume reduction surgery has been recommended by international guidelines for use in such patients. In addition, a bronchoscopic bronchopleural fistula is a disease that seriously affects the living quality and prognosis of patients, and the application of one-way valve occlusion technology is also one of the clinical treatment options. However, the related product consumables are expensive, which limits the accessibility of this technology for patients. Therefore, it is necessary to develop a corresponding medical equipment with lower research and development costs, and realize the structure and function of the one-way valve in the bronchus through independently designed solutions.

The one-way valve for bronchus currently used in clinical practice is a combination of a nickel-titanium alloy skeleton and a coating material to achieve the function of the one-way valve for bronchus with radial support force, however the manufacturing process of the one-way valve for bronchus is relatively complex and the yield is low.

### SUMMARY

The present application aims to solve at least one of the technical problems existing in the prior art or related art.

In view of this, the embodiments of the present application provide a one-way valve for bronchus, comprising:
a support portion, wherein an outer wall of the support portion is used for abutting against an inner wall of the bronchus;
an air-permeable portion that is provided in the support portion and is connected to an inner wall of the support portion;
wherein the support portion and the air-permeable portion are of an integrated structure.

In one possible implementation, the support portion and the air-permeable portion are made of a polymer material.

In one possible implementation, the one-way valve for bronchus further comprises:
a limiting portion that is formed on a partial region of the outer wall of the support portion and is used for abutting against a distal end of the bronchus.

In one possible implementation, the limiting portion comprises a threaded structure/or a pleated structure formed on the outer wall of the support portion.

In one possible implementation, the one-way valve for bronchus further comprises:
a limiting protrusion that is provided on a further partial region of the support portion and is used for abutting against the inner wall of the bronchus.

In one possible implementation, the support portion, the air-permeable portion, the limiting portion and the limiting protrusion are of an integrated structure.

In one possible implementation, there are multiple limiting protrusions, and the multiple limiting protrusions are arranged at intervals along a circumference of the support portion.

In one possible implementation, a gap is arranged between an end of the air-permeable portion and an end of the support portion.

In one possible implementation, the air-permeable portion comprises:
a flared segment connected to the outer wall of the support portion;
a narrowed segment communicated with the flared segment.

In one possible implementation, a cross-section of the narrowed segment along a height direction of the one-way valve for bronchus is rectangular, so that the air-permeable portion is fish-mouth-shaped.

Compared with the prior art, the present application at least comprises the following beneficial effects: the one-way valve for bronchus provided by the embodiments of the present application comprises a support portion and an air-permeable portion which are of an integrated structure, wherein the support portion is used for abutting against an inner wall of the bronchus, and the air-permeable portion is used for achieving one-way ventilation. Instead of the technical solution of a combination of a nickel-titanium alloy skeleton and a coating material in the prior art, since the support portion and the air-permeable portion of the one-way valve for bronchus in the embodiments of the present application are of an integrated structure, it is not necessary to further combine a coating on the skeleton, such that the process is simple, the production cost of the one-way valve for bronchus can be reduced, the yield of the one-way valve for bronchus can be increased, and the popularization and use of the one-way valve for bronchus can be facilitated.

### BRIEF DESCRIPTION OF THE DRAWINGS

The accompanying drawings described here are used to provide a further understanding of the present application and constitute a part of the same. The schematic embodiments of the present application and their descriptions are used to explain the present application and do not constitute an improper limitation of the present application.

FIG. 1 is a structural schematic diagram of a one-way valve for bronchus provided by an embodiment of the present application at one angle;

FIG. 2 is a structural schematic diagram of a one-way valve for bronchus provided by an embodiment of the present application at a further angle;

FIG. 3 is a structural schematic diagram of a one-way valve for bronchus provided by an embodiment of the present application at a further angle;

FIG. 4 is a structural schematic diagram of a one-way valve for bronchus provided by an embodiment of the present application at a further angle;

FIG. 5 is a side view of a one-way valve for bronchus provided by an embodiment of the present application;

FIG. 6 is a structural schematic diagram of a cross-section at one angle of a one-way valve for bronchus provided by an embodiment of the present application;

FIG. 7 is a structural schematic diagram of a cross-section at a further angle of a one-way valve for bronchus provided by according to an embodiment of the present application.

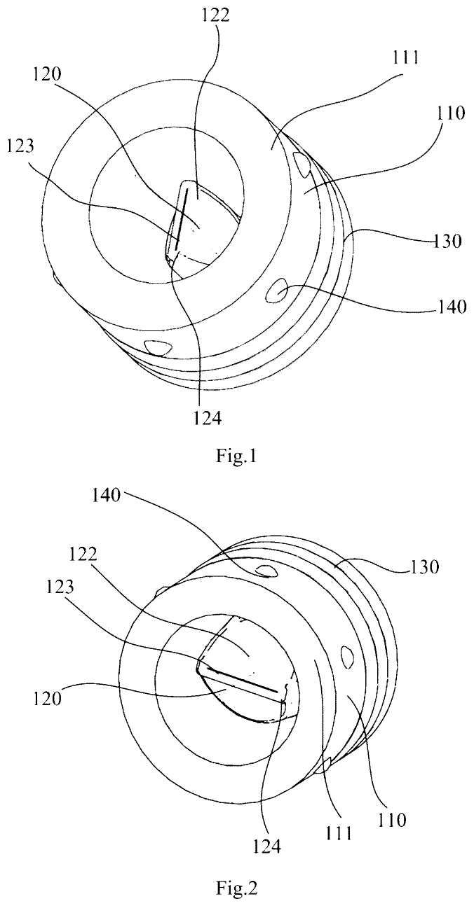

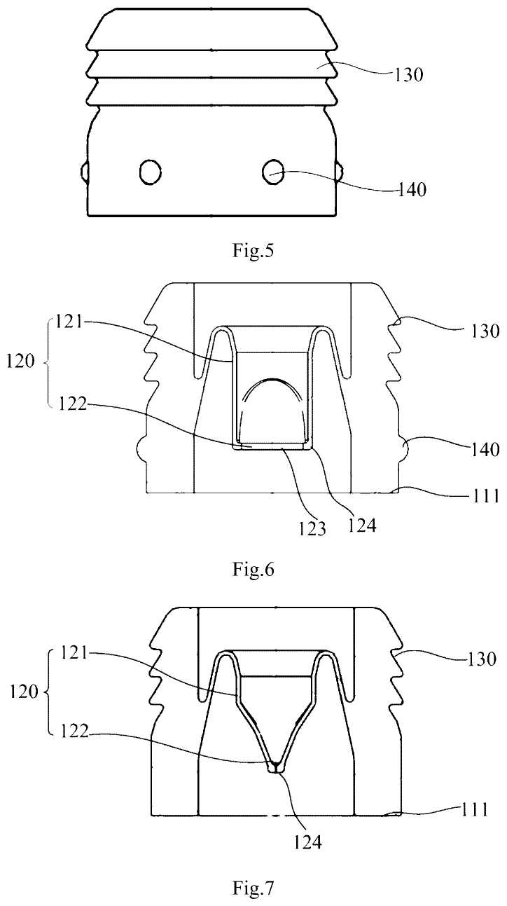

The corresponding relationships between the reference signs and the names of the components in FIGS. 1 to 7 are as follows:
110 support portion, 120 air-permeable portion, 130 limiting portion, 140 limiting protrusion;
111 end of the support portion;
121 flared segment, 122 narrowed segment; 123 valve opening; 124 end of the air-permeable portion.

### DETAILED DESCRIPTION

In order to make the objects, technical solutions and advantages of the present application more clearly, the present application will be further described in detail below with reference to the accompanying drawings and embodiments. Obviously, the described embodiments are only some, and not all, of the embodiments of the present application. All other embodiments obtained by those skilled in the art based on the embodiments of the present application fall within the scope of the present application.

As shown in FIGS. 1 and 2, an embodiment of the present application provides a one-way valve for bronchus, comprising: a support portion 110, wherein an outer wall of the support portion 110 is used for abutting against an inner wall of the bronchus; an air-permeable portion 120 provided in the support portion 110 and connected to an inner wall of the support portion 110; wherein the support portion 110 and the air-permeable portion 120 are of an integrated structure.

The one-way valve for bronchus provided by the embodiment of the present application comrpises a support portion 110 and an air-permeable portion 120 which are of an integrated structure, wherein the support portion 110 is used for abutting against an inner wall of the bronchus, and the air-permeable portion 120 is used for achieving one-way ventilation. Instead of the technical solution of a combination of a nickel-titanium alloy skeleton and a coating material in the prior art, since the support portion 110 and the air-permeable portion 120 of the one-way valve for bronchus in the embodiment of the present application are of an integrated structure, it is not necessary to further combine a coating on the skeleton, such that the process is simple, the production cost of the one-way valve for bronchus can be reduced, the yield of the one-way valve for bronchus can be increased, and the popluarization and use of the one-way valve for bronchus can be facilitated.

In one possible implementation, the support portion 110 and the air-permeable portion 120 are made of a polymer material.

In this technical solution, the material for the support portion 110 and the air-permeable portion 120 is further provided. The support portion 110 and the air-permeable portion 120 are made of a polymer material, such that on the one hand, the strength of the one-way valve for bronchus can be increased, and on the other hand, the service life of the one-way valve for bronchus can be increased.

As shown in FIGS. 1 to 3, in one possible implementation, the one-way valve for bronchus further comprises a limiting portion 130 that is formed in a partial region of the outer wall of the support portion 110, and is used for abutting against a distal end of the bronchus, that is, an end of the bronchus close to an alveoli.

In this technical solution, the one-way valve for bronchus can further comprises a limiting portion 130 that is provided on the outer wall of the support portion 110, and is used for abutting against a distal end of the bronchus, which is conducive to embedding with the airway wall, prevents displacement, and forms a good occlusion effect.

In one possible implementation, the limiting portion 130 comprises a threaded structure and/or a pleated structure formed on the outer wall of the support portion 110. In one specific embodiment, as shown in FIGS. 6 and 7, on an axial section of the one-way valve for bronchus, the limiting portion 130 is of a toothed structure formed on the outer wall of the support portion 110.

In this technical solution, the style of the limiting portion 130 is further provided. The limiting portion 130 comprises a threaded structure and/or a pleated structure, such that the limiting portion 130 can better abuts against the distal end of the bronchus, which is conducive to embedding with the airway wall, prevents displacement, and forms a good occlusion effect.

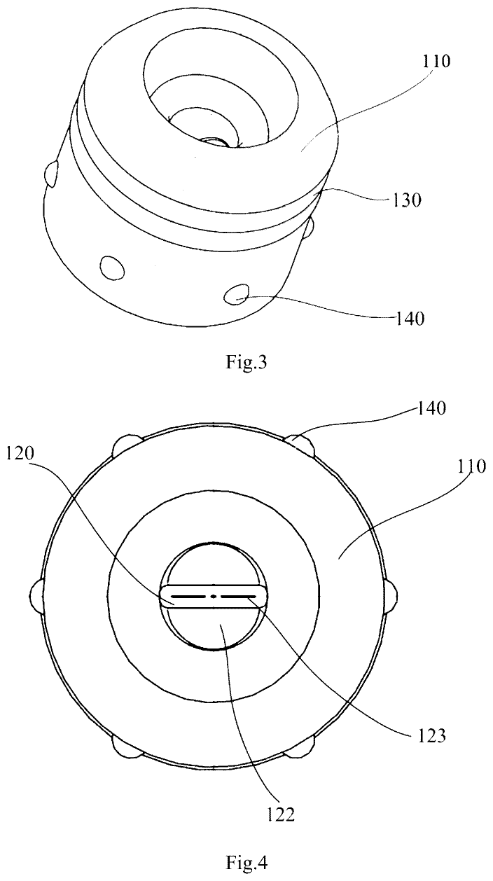

In one specific embodiment, as shown in FIG. 3 and FIG. 5 to FIG. 7, the limiting portion 130 comprises a multi-layer pleated structure, and an outer periphery of the pleated structure is an inclined surface to form an inverted buckle, thereby facilitating embedding with the airway wall.

As shown in FIGS. 1 to 6, in one possible implementation, the one-way valve for bronchus further comprises a limiting protrusion 140 provided on a further partial region of the support portion 110. The limiting protrusion 140 is used for abutting against the inner wall of the bronchus. That is to say, a partial region of the support portion 110 in which the limiting portion 130 is not provided is further provided with the limiting protrusion 140.

In this technical solution, in addition to the limiting portion 130, a limiting protrusion 140 can be further provided on the outer wall of the support portion 110. The limiting protrusion 140 abuts against the inner wall of the bronchus to ensure that the entire one-way valve for bronchus is tightly fitted and fixed to the airway.

As shown in FIG. 6 and FIG. 7, in one possible implementation, the support portion 110, the air-permeable portion 120, the limiting portion 130 and the limiting protrusion 140 are of an integrated structure.

In this technical solution, the support portion 110, the air-permeable portion 120, the limiting portion 130 and the limiting protrusion 140 are of an integrated structure, such that the structure of the one-way valve for bronchus can be simplified and the production and processing of the one-way valve for bronchus is facilitated.

In one specific embodiment, as shown in FIG. 6 and FIG. 7, the support portion 110 is of a hollow columnar structure, the air-permeable portion 120 is formed inside the support portion 110, and a valve opening 123 is provided at one end of the air-permeable portion 120. The limiting portion 130 and the limiting protrusion 140 are formed on the outer wall of the support portion 110, respectively. In a height direction of the one-way valve for bronchus, the limiting portion 130 is located on a region of the support portion 110 away from the valve opening 123, and the limiting protrusion 140 is located on a region of the support portion 110 close to the valve opening 123.

As shown in FIG. 1 to FIG. 6, in one possible implementation, there are multiple limiting protrusions 140, and the multiple limiting protrusions 140 are arranged at intervals along a circumference of the support portion 110.

In this technical solution, there are multiple limiting protrusions 140, and the multiple limiting protrusions 140 abut against the inner wall of the bronchus, which can further ensure that the entire one-way valve for bronchus is tightly fitted and fixed to the airway.

As shown in FIGS. 1, 2, 6 and 7, in one possible implementation, in the height direction of the one-way valve for bronchus, a gap is arranged between an end 124 of the air-permeable portion 120 and an end 111 of the support portion 110, so that a plane where the valve opening 123 of the air-permeable portion 120 is located is slightly lower than a plane of a tube opening of the support portion 110 with the limiting protrusion 140.

This arrangement can provide a remedial treatment for patients of treatment failing due to false-negative collateral ventilation in the target lung lobe; based on a space between the end 124 of the air-permeable portion 120 and the end 111 of the support portion 110, so as to solve the problem in the art that the one-way valve in the bronchus can only be removed once treatment fails due to false-negative target lobe bypass ventilation. This can increase the success rate of the surgery and reduce the pain of the patients.

As shown in FIG. 6 and FIG. 7, in one possible implementation, the air-permeable portion 120 comprises a flared segment 121 connected to the outer wall of the support portion 110; and a narrowed segment 122 communicated with the flared segment 121.

In this technical solution, the style of the air-permeable portion 120 is further provided. The air-permeable portion 120 comprises a flared segment 121 and a narrowed segment 122. Gas can flow from the flared segment 121 to the narrowed segment 122, but cannot flow from the narrowed segment 122 to the flared segment 121, and thus a one-way ventilation of the air-permeable portion 120 is achieved.

Further, as shown in FIG. 6 and FIG. 7, the valve opening 123 is located in the narrowed segment 122, and one end of the flared segment 121 away from the valve opening 123 is folded outward and extends toward the valve opening 123 to be connected to the support portion 110.

As shown in FIG. 6, in one possible implementation, a cross-section of the narrowed segment 122 along the height direction of the one-way valve for bronchus is rectangular, so that the air-permeable portion 120 is fish-mouth-shaped.

Specifically, as shown in FIG. 6, in the height direction of the one-way valve for bronchus, a distance between two opposite sides of the narrowed segment 122 remains unchanged along a direction away from the flared segment 121, and as shown in FIG. 7, a distance between the other two opposite sides of the narrowed segment 122 is decreasing along the direction away from the flared segment 121, so that as shown in FIGS. 1 and 2, the air-permeable portion 120 as a whole presents a flat fish-mouth-shaped structure.

In this technical solution, the shape of the air-permeable portion 120 is further provided. The air-permeable portion 120 is fish-mouth-shaped, which can improve the air permeability. At the same time, the fish-mouth-shaped air-permeable portion 120 can better cooperate with the support portion 110.

The one-way valve for bronchus provided by the embodiments of the present application can be used for a bronchoscopic lung volume reduction (BLVR) surgery and a bronchoscopic closure of bronchopleural fistula. Specifically, the present application provides a one-way valve for bronchus that can be used for a bronchoscopic lung volume reduction and a bronchoscopic closure of bronchopleural fistula, which is of an integrated structure, and comprises a support portion 110 formed by a polymer material skeleton structure and an air-permeable portion 120 with a one-way valve structure. One end of the support portion 110 is threaded, which is used to be inserted into a distal end of the bronchus and embedded with the airway wall, and the other end is cylindrical, and its outer surface has a protrusion to ensure that the entire structure is tightly fitted and fixed to the airway. There is an integrated one-way valve structure in the skeleton structure, which is of a fish-mouth-like diagonal structure, allowing unidirectional passage of gas.

In the present application, the terms “first”, “second”, and “third” are intended for purposes of the description only and should not be understood as indicating or implying relative importance; the term “multiple” refers to two or more, unless otherwise specified. The terms such as “installed”, “connected with”, “connected to”, and “fixed” should be understood in a broad sense. For example, the term “connected to” can mean a fixed connection, a detachable connection, or an integral connection; the term “connected with” can mean a direct connection or an indirect connection through an intermediate medium. For those skilled in the art, specific meanings of the above terms in the present application can be understood according to the specific situation.

In the description of the present application, it should be understood that the orientations or positional relationships indicated by the terms “above”, “below”, “left”, “right”, “front”, “back”, and the like are based on the orientations or positional relationships shown in the accompanying drawings, and are only for the convenience of describing the present application and simplifying the description, rather than indicating or implying that the device or unit referred to must have a specific direction, be constructed and operated in a specific orientation, and therefore, the terms should not be understood as a limitation on the present application.

In the description of this specification, the description of the terms “one embodiment”, “some embodiments”, “specific embodiment”, and the like means that specific features, structures, materials or characteristics described in combination with the embodiment or example are comprised in at least one embodiment or example of the present application. In this specification, it is not necessary for the schematic representation of the above terms to refer to the same embodiment or example. Moreover, the specific features, structures, materials or characteristics described can be combined in any one or more embodiments or examples in a suitable manner.

The above are only preferred embodiments of the present application and are not intended to limit the present application. For those skilled in the art, the present application may have various modifications and variations. Any modification, equivalent replacement, improvement, and the like made within the spirit and principle of the present application shall be comprised within the scope of the present application.

1. A one-way valve for bronchus, wherein the one-way valve for bronchus comprises:
a support portion, wherein an outer wall of the support portion is used for abutting against an inner wall of the bronchus;
an air-permeable portion that is provided in the support portion and is connected to an inner wall of the support portion;
wherein the support portion and the air-permeable portion are of an integrated structure.

2. The one-way valve for bronchus according to claim 1, wherein,
the support portion and the air-permeable portion are made of a polymer material.

3. The one-way valve for bronchus according to claim 1, wherein the one-way valve for bronchus further comprises:
a limiting portion that is formed on a partial region of the outer wall of the support portion and is used for abutting against a distal end of the bronchus.

4. The one-way valve for bronchus according to claim 3, wherein,
the limiting portion comprises a threaded structure/or a pleated structure formed on the outer wall of the support portion.

5. The one-way valve for bronchus according to claim 3, wherein the one-way valve for bronchus further comprises:
a limiting protrusion that is provided on a further partial region of the support portion and is used for abutting against the inner wall of the bronchus.

6. The one-way valve for bronchus according to claim 5, wherein,
the support portion, the air-permeable portion, the limiting portion and the limiting protrusion are of an integrated structure.

7. The one-way valve for bronchus according to claim 5, wherein,
there are multiple limiting protrusions, and the multiple limiting protrusions are arranged at intervals along a circumference of the support portion.

8. The one-way valve for bronchus according to claim 1, wherein,
a gap is arranged between an end of the air-permeable portion and an end of the support portion.

9. The one-way valve for bronchus according to claim 1, wherein the air-permeable portion comprises:
a flared segment connected to the outer wall of the support portion;
a narrowed segment communicated with the flared segment.

10. The one-way valve for bronchus according to claim 9, wherein,
a cross-section of the narrowed segment along a height direction of the one-way valve for bronchus is rectangular, so that the air-permeable portion is fish-mouth-shaped.
